(19) United States
(12) Patent Application Publication
(10) Pub. No.: US 2006/0015103 A1
(43) Pub. Date: Jan. 19, 2006

(54) I-beam configuration bone plate

(76) Inventor: Shawn Burke, Jacksonville, FL (US)

Correspondence Address: ROGERS TOWERS, P.A., 1301 RIVERPLACE BOULEVARD, SUITE 1500, JACKSONVILLE, FL, 32207, US

(21) Appl. No.: 11/185,376

(22) Filed: Jul. 19, 2005

Related U.S. Application Data
(60) Provisional application No. 60/589,091, filed on Jul. 19, 2004.

(51) Int. Cl. A61F 2/30

(52) U.S. Cl. 606/69

(57) ABSTRACT

A bone plate having a first opposing end containing screw-receiving apertures and a second opposing send containing screw-receiving apertures, the first opposing end and the second opposing end being joined by a plurality of bridging members, the bridging members having an I-beam configuration in cross-section and defining at least one opening in the main body of the bone plate.

This application claims the benefit of U.S. Provisional Patent Application Ser. No. 60/589,091, filed Jul. 19, 2004.

### BACKGROUND OF THE INVENTION

This invention relates generally to the field of bone plate fixation devices that are used to secure bones or separated bone segments in a rigid and fixed relationship, usually so that osteosynthesis or bone regeneration may occur.

Orthopedic fixation devices in the form of flat or slightly curved plates are well known and are used to fix and secure adjacent bones or bone segments in a rigid manner, such as for example to maintain adjacent vertebrae in a fixed manner relative to each other, or to maintain fractured or surgically cut bone segments in a fixed manner relative to each segment such that bone regeneration may occur. One common application is to apply a bone plate to a sternum that has been cut in order to provide access to the heat, for example. The bone plates are typically composed of a biocompatible, solid-body, metal plate having a generally rectangular configuration. Plural screw-receiving apertures are positioned adjacent opposing ends of the plate, such that bone screws may be inserted and threaded into the bone material to secure the plate in position. The bone plates must be of sufficient thickness to resist bending or flexing, as it is imperative that the adjacent bones or bone segments remain in a fixed relationship.

Because the bone plates are disposed internally within the human body, it is most desirable that the overall size and mass of the bone plate be minimized without sacrificing strength and rigidity. There are also situations where it may be desirable to provide visual or physical access to an area that underlies the bone plate. This can be addressed by providing open spaces within the body of the bone plate, but the amount of open area is limited by the need to retain sufficient strength and rigidity in the main body.

It is an object of this invention to address the desire for reduced mass, increased visibility and increased access in a bone fixation plate by providing a new and improved structure for a bone plate wherein the overall mass of the bone plate is reduced without reducing strength and rigidity characteristics and wherein open areas of increased size are provided within the interior of the bone plate to allow for visual or physical access.

### SUMMARY OF THE INVENTION

The invention comprises in general a bone fixation plate composed of a biocompatible material that is suitable for securing together in a fixed and rigid manner two bones or bone segments such that relative movement between the two bones or bone segments is precluded, such as for example the fixation of adjacent vertebrae, or the fixation of adjacent bone segments when a bone has been fractured, or the fixation of adjacent bone segments when a bone has been surgically severed during a medical procedure.

The bone plate is typically composed of metal, although other materials possessing sufficient strength and rigidity may also be suitable, and comprises opposing screw-receiving apertures, preferably in plural sets, adapted to receive bone screws for securing the bone plate to the adjacent bones or bone segments. The bone plate body is a relatively thin member with length and width dimensions greatly exceeding the thickness dimension, typically flat or slightly curved depending on the application, and comprises at least one bridging member that extends between the opposing ends of the bone plate containing the screw-receiving apertures and spanning members that extend between the screw-receiving apertures on each of the opposing ends of the bone plate. Preferably, the bone plate body comprises a plurality of bridging members, such that the bridging members define one or more interior openings within the bone plate body and/or one or more lateral recesses. One or preferably all of the bridging members have an I-beam configuration in cross-section, such that each bridging member comprises a pair of generally parallel surface members joined by a generally perpendicularly disposed bracing member. Most preferably, the spanning members are constructed having an I-beam or a half I-beam configuration as well. In this manner, the overall mass of the bone plate and the area obscured by the bone plate are reduced without reducing the strength and rigidity of the bone plate in comparison to a bone plate having a solid body.

### BRIEF DESCRIPTION OF THE DRAWINGS

FIG. 1 is a top view of the surface of an embodiment of the I-beam configuration bone plate invention.

FIG. 2 is a side view showing an embodiment having a slight curvature of the body.

FIG. 3 is a cross-sectional view taken along line III-III of FIG. 1 showing the I-beam configuration of the bridging members.

FIG. 4 is a cross-sectional view taken along line IV-IV of FIG. 1 showing the half I-beam configuration of the spanning members.

FIG. 5 is a top view of an alternative embodiment of the invention.

### DETAILED DESCRIPTION OF THE INVENTION

With reference to the drawings, the invention will now be described with regard for the best mode and the preferred embodiment. In general, the invention comprises a bone fixation plate composed of a biocompatible material that is suitable for securing together in a fixed and rigid manner two bones or bone segments such that relative movement between the two bones or bone segments is precluded, such as for example the fixation of adjacent vertebrae, or the fixation of adjacent bone segments when a bone has been fractured, or the fixation of adjacent bone segments when a bone has been surgically severed during a medical procedure. For ease of reference in this disclosure, the term “bone segment” shall be taken to include bones, bone fragments, bone sections or the like.

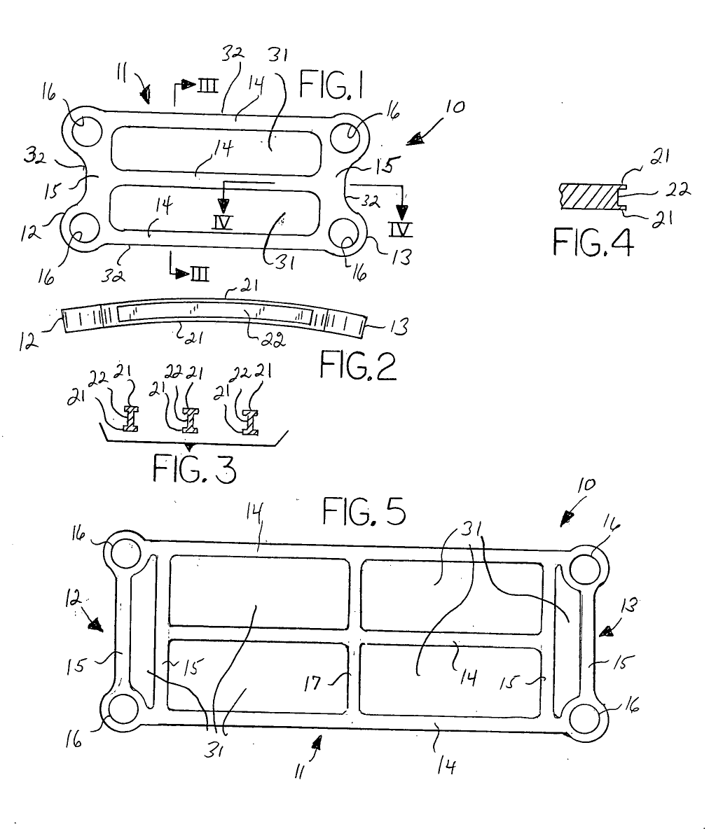

The bone plate 10 is a generally flat, as shown in FIG. 1, or curved member, as shown in FIG. 2, composed of a metal or similar biocompatible material having good strength and rigidity characteristics, such that the bone plate 10 will not excessively flex or bend under stresses expected to be encountered during use. The bone plate 10 may have various peripheral shapes, and is shown in representative FIG. 1 as being generally rectangular. The length and width dimensions may vary dependent on intended use, to the extent for example that the bone plate 10 may be generally square or elongated with the length dimension greater than the width dimension, the length dimension being defined herein to be the dimension extending in the direction between the sets of screw-receiving apertures 16 on the opposing ends 12 and 13 of the body 11.

The bone plate 10 comprises a main body 11 bounded on opposing ends by first opposing end 12 and second opposing end 13. The opposing ends 12 and 13 are connected by at least one but most preferably a plurality of generally elongated bridging members 14, with the bridging members 14 defining one or more relatively large openings 31 within the main body 11 and/or exterior recesses 32. At least one and most preferably a plurality of screw-receiving apertures 16 are disposed in the first end 12 of main body 11. Another set of at least one and most preferably a plurality of screw-receiving apertures 16 are disposed in the second end 13 of the main body 11. The main body 11 further preferably comprises spanning members 15 defining the ends 12 and 13 by extending generally perpendicularly to the bridging members 14 and between the screw-receiving apertures 16.

In order to provide sufficient rigidity and strength in bone plate 10, the bridging members 14 are constructed to have an I-beam configuration when taken in cross-section, as shown in FIG. 3, such that each bridging member 14 comprises a pair of surface or flange members 21 in generally parallel orientation joined by a perpendicularly oriented bracing or web member 22. This beam structure is a well-known engineering construct that is highly resistant to bending from load and shear forces. In like manner, it is preferable that the spanning members 15 be constructed to have an I-beam configuration in cross-section if possible, or at least to have a half I-beam configuration in cross-section as shown in FIG. 4, wherein the outer side of the spanning member 15 has a pair of outwardly extending flange members 21 and the outer wall of the spanning member 15 is somewhat akin to a web member 22.

In the alternative embodiment shown in FIG. 5, a larger bone plate 10 is shown. The bridging members 14 are provided with additional support in the form of an interior spanning member 17 of I-beam configuration. The ends 12 and 13 are each structured as a pair of spanning members 15 of I-beam configuration, with an interior opening 31 being defined in each end 12 and 13.

Utilizing the I-beam configuration results in a main body 11 that is at least equal in resistance to bending, flexing, load and shear to that of a body plate composed of solid material of much greater mass. Utilization of the I-beam configuration for the bridging members 14 creates openings 31 for visual or physical access to the area spanned by the bone plate 10 for ease of manufacture, the bone plate 10 may be machined or cast as a two-piece member in upper and lower halves, then joined by welding or other suitable techniques to form a unified member.

It is contemplated that equivalents and substitutions for certain elements set forth above may be obvious to those skilled in the art, and therefore the true scope and definition of the invention is to be as set froth in the following claims.

I claim:

1. A bone fixation plate comprising a main body having a first opposing end containing screw-receiving apertures and a second opposing end containing screw-receiving apertures, said first opposing end and said second opposing end joined by a plurality of bridging members, said bridging members having an I-beam configuration in cross-section.

2. The device of claim 1, wherein said bridging members define at least one interior opening in said main body.

3. The device of claim 2, wherein said bridging members further define at least one exterior recess in said main body.

4. The device of claim 1, wherein said first and said second opposing ends comprise spanning members.

5. The device of claim 4, said spanning members having an I-beam configuration in cross-section.

6. The device of claim 5, wherein each said first and said second opposing ends have at least two spanning members defining an interior opening.

7. The device of claim 1, wherein said main body is curved.

8. The device of claim 2, wherein said first and said second opposing ends comprise spanning members.

9. The device of claim 8, said spanning members having an I-beam configuration in cross-section.

10. A bone fixation plate comprising a main body having a first opposing end containing screw-receiving apertures and a second opposing end containing screw-receiving apertures, said first opposing end and said second opposing end joined by a plurality of bridging members, said bridging members comprising a web member extending between two generally parallel flange members.

11. The device of claim 10, wherein said bridging members define at least one interior opening in said main body.

12. The device of claim 11 wherein said bridging members further define at least one exterior recess in said main body.

13. The device of claim 10, wherein said first and said second opposing ends comprise spanning members.

14. The device of claim 13, said spanning members comprising a web member extending between two generally parallel flange members.

15. The device of claim 14, wherein each said first and said second opposing ends have at least two spanning members defining an interior opening.

16. The device of claim 10, wherein said main body is curved.

17. The device of claim 11, wherein said first and said second opposing ends comprise spanning members.

18. The device of claim 17, said spanning members comprising a web member extending between two generally parallel flange members.
